(19) United States
(12) Patent Application Publication
(10) Pub. No.: US 2026/0008735 A1
(43) Pub. Date: Jan. 8, 2026

(54) METHANOL SYNTHESIS SYSTEM USING BIOMASS AND METHOD FOR CONTROLLING THE SAME

(71) Applicants: HYUNDAI MOTOR COMPANY, Seoul (KR); KIA CORPORATION, Seoul (KR)

(72) Inventor: Dong Seok Ko, Hwaseong-si (KR)

(73) Assignees: HYUNDAI MOTOR COMPANY, Seoul (KR); KIA CORPORATION, Seoul (KR)

(21) Appl. No.: 18/956,671

(22) Filed: Nov. 22, 2024

(30) Foreign Application Priority Data
Jul. 5, 2024 (KR) 10-2024-0089029

(51) Int. Cl. C07C 29/152; C12M 1/00; C12M 1/107; C12P 5/02

(52) U.S. Cl. CPC C07C 29/152; C12M 21/04; C12M 47/18; C12P 5/023

(57) ABSTRACT

A methanol synthesis system which includes a first producing device to produce methane and carbon dioxide by introducing biomass,
a second producing device to produce hydrogen and carbon dioxide by allowing the methane, which is supplied from the first producing device, to react with steam, a first controller to control a mole flow rate of the carbon dioxide supplied from the first producing device, a second controller to control a mole flow rate of the hydrogen supplied from the second producing device, and a methanol synthesizing device to synthesize methanol by allowing the carbon dioxide supplied from the first controller, the hydrogen supplied from the second controller, and the carbon dioxide supplied from the second producing device to react with each other.

### CROSS-REFERENCE TO RELATED APPLICATION

This application claims the benefit of priority to Korean Patent Application No. 10-2024-0089029, filed in the Korean Intellectual Property Office on Jul. 5, 2024, the entire contents of which are incorporated herein by reference.

### TECHNICAL FIELD

The present disclosure relates to a system that synthesize methanol from biomass and a method for controlling the same.

### BACKGROUND

Recently, as the concern about the depletion of energy resources and environmental pollution caused by excessive use of fossil fuel is increased, the demand for energy sources based on non-fossil fuel, renewable, sustainable, and environmental-friendly has been rapidly increased. As one example, biogas has recently been spotlighted as an energy source that does not rely on fossil fuels.

Biogas is a renewable and available energy source that can be used for heating, electricity, and many other applications. It is a gas mixture typically produced by decomposing organic matter in the absence of oxygen. Specifically, biogas may be produced by decomposing unfertilized organic substances (i.e., biomass) such as agricultural waste, manure, household waste, a plant material, sewage, green waste, or food waste through an anaerobic digestion reaction. The biogas may mainly contain methane (CH₄) and carbon dioxide (CO₂), and may have a small amount of other substances (e.g., contaminants, impurities, “foreign substances” or components other than methane and carbon dioxide) hydrogen sulfide (H₂S), moisture, siloxane, and other possible components.

The biogas is a key basic material for various and complex chemical synthesis reactions, including fuel for transportation, and is used as a material for synthesizing methanol (CH₃OH), but one or more side reactions may occur in the process of synthesizing methanol (CH₃OH). Accordingly, there is a need for systems and methods for controlling such systems, that are capable of preventing or reducing undesired side reactions, and that control the optimal operation of a methanol synthesis system that utilizes biomass.

### SUMMARY

The present disclosure addresses at least the above-mentioned problems that are known in the prior art, while also preserving the advantages associated with biomass-based methanol production.

An aspect of the present disclosure provides a methanol synthesis system, capable of controlling the optimal condition for synthesizing methanol (CH₃OH) using biomass, and a method for controlling the same.

The technical problems to be solved by the present disclosure are not limited to the aforementioned problems, and any other technical problems not mentioned herein will be clearly understood from the following description by those skilled in the art to which the present disclosure pertains.

(1) The present disclosure provides a methanol synthesis system which includes a first producing device to produce methane and carbon dioxide by introducing biomass, a second producing device to produce hydrogen and carbon dioxide by allowing the methane, which is supplied from the first producing device, to react with steam, a first controller to control a mole flow rate of the carbon dioxide supplied from the first producing device, a second controller to control a mole flow rate of the hydrogen supplied from the second producing device, and a methanol synthesizing device to synthesize the methanol by allowing the carbon dioxide supplied from the first controller, the hydrogen supplied from the second controller, and the carbon dioxide supplied from the second producing device to react with each other.

(2) The first producing device provides an anaerobic digestion device to produce biogas by performing an anaerobic digestion process for the biomass, a preprocessor to remove a foreign substance from the biogas, and a first separator to separate the methane and the carbon dioxide from the biogas having no foreign substance, as the foreign substance is removed from the biogas in the preprocessor in (1).

(3) The methane separated in the first separator has purity of at least 95% in (1) or (2).

(4) There is provided a methanol synthesis system in which the second producing device includes a reforming reactor to allow the steam and a portion of the methane supplied from the first producing device to react with each other, a heat supplier to supply heat to the reforming reactor by using, as fuel, a remaining portion of the methane supplied from the first producing device, a shift converter to produce steam reforming gas by allowing a product from the reforming reactor to react with the steam, a second separator to separate hydrogen from the steam reforming gas, and a third separator to separate carbon dioxide from the steam reforming gas having no hydrogen, as the hydrogen is separated from the steam reforming gas in the second separator in any one of (1) to (3).

(5) There is provided, in any one of (1) to (4), a methanol synthesis system, in which the reforming reactor produces carbon monoxide and hydrogen by allowing the portion of the methane supplied from the first producing device and the steam to react with each other through following Chemical Equation 1, and the shift converter produces carbon dioxide and hydrogen by allowing the carbon monoxide and the hydrogen produced from the reforming reactor to react with each other through following Chemical Equation 2,

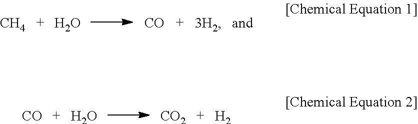

(6) There is provided a methanol synthesis system in which the hydrogen separated from the second separator has purity of at least 99%, in any one of (1) to (5).

(7) There is provided, in any one of (1) to (6), a methanol synthesis system in which a mole flow rate of the carbon dioxide received from the first producing device to the methanol synthesizing device and a mole flow rate of the hydrogen supplied from the second producing device to the methanol synthesizing device are controlled in a first mode, and the first mode is to determine the mole flow rate of the carbon dioxide controlled by the first controller through Equation 1, and to determine the flow rate of the hydrogen controlled by the second controller to a whole mole flow rate of the hydrogen supplied from the second producing device,

$$[ {Feed}_{{CO}_{2}} ] = \frac{[ {H}_{2} ]}{[ Eq ]} - [ C {O}_{2} ] , \qquad [ Equation 1 ]$$

in which in Equation 1,
[Feed_{CO}_{₂}] denotes the mole flow rate of the carbon dioxide supplied to the methanol synthesizing device,
[H₂] denotes the mole flow rate of the hydrogen supplied from the second producing device,
[CO₂] denotes the mole flow rate of the carbon dioxide supplied from the second producing device, and
[Eq] denotes an arbitrary value input by a user (8). There is provided a methanol synthesis system in which the methanol synthesizing device synthesizes the methanol by allowing the carbon dioxide supplied from the first controller, the hydrogen supplied from the second controller, and the carbon dioxide supplied from the second producing device to react with each other through Chemical Equation 4, and [Eq] in Equation 1 is in a range 3 to 6,

CO₂+3H₂→CH₃OH+H₂O [Chemical Equation 4]

in any one of (1) to (7).

(9) There is provided a methanol synthesis system in which the mole flow rate of the carbon dioxide received from the first producing device to the methanol synthesizing device and the mole flow rate of the hydrogen supplied from the second producing device to the methanol synthesizing device are controlled depending on a second mode, and the second mode is to determine the mole flow rate of the carbon dioxide controlled by the first controller to a value of ‘0’, and to determine the mole flow rate of the hydrogen controlled by the second controller through following Equation 2,

[Feed_{H}_{₂}]=[CO₂]×[Eq], [Equation 2]

in which in Equation 2,
[Feed_{H}_{₂}] denotes the mole flow rate of the hydrogen supplied to the methanol synthesizing device,
[CO₂] denotes the mole flow rate of the carbon dioxide supplied from the second producing device, and
[Eq] denotes an arbitrary value input by a user, in any one of (1) to (8).

(10) There is provided a methanol synthesis system, in which the methanol synthesizing device synthesizes the methanol by allowing the carbon dioxide supplied from the first controller, the hydrogen supplied from the second controller, and the carbon dioxide supplied from the second producing device to react with each other through following Chemical Equation 4, and [Eq] in Equation 1 is in a range 3 to 6,

CO₂+3H₂→CH₃OH+H₂O(ΔH=−49.6 kJ/mol) [Chemical Equation 4]

in any one of (1) to (9).

(11) There is provided a methanol synthesis system in which the methanol synthesizing device synthesizes methanol by allowing the carbon dioxide supplied from the first controller, the hydrogen supplied from the second controller, and the carbon dioxide supplied from the second producing device to react with each other through following Chemical Equation 4,

CO₂+3H₂→CH₃OH+H₂O(ΔH=−49.6 kJ/mol), [Chemical Equation 4]

in any one of (1) to (10).

(12) There is provided a methanol synthesis system in which reaction in Chemical Equation 4 is performed at a temperature of 250° C. under at least 50 atmospheric pressure in presence of copper catalyst, in any one of (1) to (11).

(13) There is provided a methanol synthesis system in which reaction in Chemical Equation 4 is performed at a temperature of 150° C. under at least 30 atmospheric pressure in presence of platinum catalyst, in any one of (1) to (12).

(14) There is provided a method for controlling the methanol synthesizing device according to any one of (1) to (13), in which the methanol synthesizing device is controlled in a first mode in which a mole flow rate of carbon dioxide, which is controlled by the first controller, is determined through following Equation 1, and a mole flow rate of hydrogen controlled by the second controller is determined to a whole mole flow rate of hydrogen supplied from the second producing device, or in a second mode, in which the mole flow rate of the carbon dioxide controlled by the first controller is determined to a value of ‘0’, and the mole flow rate of the hydrogen controlled by the second controller is determined through following Equation 2,

$$[ {Feed}_{{CO}_{2}} ] = \frac{[ {H}_{2} ]}{[ Eq ]} - [ C {O}_{2} ] , \qquad [ Equation 1 ]$$

in which in Equation 1,
[Feed_{CO}_{₂}] denotes the mole flow rate of the carbon dioxide supplied to the methanol synthesizing device,
[H₂] denotes the mole flow rate of the hydrogen supplied from the second producing device,
[CO₂] denotes the mole flow rate of the carbon dioxide supplied from the second producing device, and
[Eq] denotes an arbitrary value input by a user, and

[Feed_{H}_{₂}]=[CO₂]×[Eq], [Equation 2]

in which in Equation 2,
[Feed_{H}_{₂}] denotes the mole flow rate of the hydrogen supplied to the methanol synthesizing device,
[CO₂] denotes the mole flow rate of the carbon dioxide supplied from the second producing device, and
[Eq] denotes an arbitrary value input by a user.

(15) There is provided a method for controlling the methanol synthesizing device, in which the methanol synthesizing device synthesizes the methanol by allowing the carbon dioxide supplied from the first controller, the hydrogen supplied from the second controller, and the carbon dioxide supplied from the second producing device to react with each other through following Chemical Equation 4, and [Eq] in Equations 1 and 2 is in a range 3 to 6,

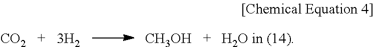

The disclosure thus provides, in various aspects and embodiments, a system for methanol synthesis, and related production and control methods, comprising: a first reaction system configured to receive biomass from a source and react the biomass under conditions to produce methane and carbon dioxide; a second reaction system in fluid communication with the first reaction system and configured (i) to receive methane supplied from the first reaction system and, (ii) to react the methane with steam under conditions produce hydrogen and carbon dioxide; a first controller configured to control a mole flow rate of the carbon dioxide from the first reaction system; a second controller configured to control a mole flow rate of the hydrogen from the second reaction system; and a methanol synthesizing system in fluid communication with each of the first reaction system and the second reaction system, and configured (i) to receive the carbon dioxide produced in the first reaction system, (ii) to receive the hydrogen and carbon dioxide produced in the second reaction system, and (iii) to synthesize methanol from reaction between the hydrogen and the carbon dioxide; wherein the first controller controls the mole flow rate of the carbon dioxide between the first reaction system and the methanol synthesizing system, and wherein the second controller controls the mole flow rate of the hydrogen between the second reaction system and the methanol synthesizing system.

### BRIEF DESCRIPTION OF THE DRAWINGS

The above and other objects, features and advantages of the present disclosure will be more apparent from the following detailed description taken in conjunction with the accompanying drawings:

FIG. 1 is a view schematically illustrating the structure of a methanol synthesis system, according to an embodiment of the present disclosure;

FIG. 2 is a view schematically illustrating the structure of a first controller and a second controller in a methanol synthesis system, according to an embodiment of the present disclosure;

FIG. 3 is a view illustrating a methanol synthesis system controlled in a first mode, according to an embodiment of the present disclosure; and

FIG. 4 is a view illustrating a methanol synthesis system controlled in a second mode, according to an embodiment of the present disclosure.

### DETAILED DESCRIPTION

Hereinafter, the present disclosure will be described in more detail for the understanding of the present disclosure. In this case, terms and words used in the present specification and the claims shall not be interpreted as commonly-used dictionary meanings, but shall be interpreted as to be relevant to the technical scope of the present disclosure based on the fact that the present disclosure may properly define the concept of the terms to explain the invention in best ways.

The terms used in the present disclosure are provided only for the illustrative purpose, and the present disclosure is not limited thereto. The singular forms are intended to include the plural forms unless the context clearly indicates otherwise.

In this specification, It will be further understood that the terms “comprises,” “includes,” or “has,” specify the presence of stated features, numbers, steps, components, parts, or the combination thereof, but do not preclude the presence or addition of one or more other features, numbers, steps, components, and/or the combination thereof.

Inventors of the present disclosure complete the present disclosure by finding out a synthesis system, and a method for controlling the same, capable of controlling a mole flow rate of carbon dioxide, which is supplied for synthesizing methanol, properly, as it is find that a side reaction is caused due to an insufficient mole flow rate of carbon dioxide which is supplied for synthesizing methanol, when hydrogen is produced using methane produced from biomass, and when methanol is synthesized using the produced hydrogen.

### Methanol Synthesis System

The present disclosure provides a methanol synthesis system.

According to an embodiment of the present disclosure, the methanol synthesis system may at least includes a first producing device to produce methane (CH₄) and carbon dioxide (CO₂) by introducing biomass, a second producing device to produce hydrogen (H₂) and carbon dioxide by allowing methane, which is supplied from the first producing device, to react with steam (H₂O), a first controller to control a mole flow rate of carbon dioxide supplied from the first producing device, a second controller to control a mole flow rate of hydrogen supplied from the second producing device, and a methanol synthesizing device to synthesize methanol (CH₃OH) by allowing carbon dioxide supplied from the first controller, hydrogen supplied from the second controller, and carbon dioxide supplied from the second producing device to react with each other.

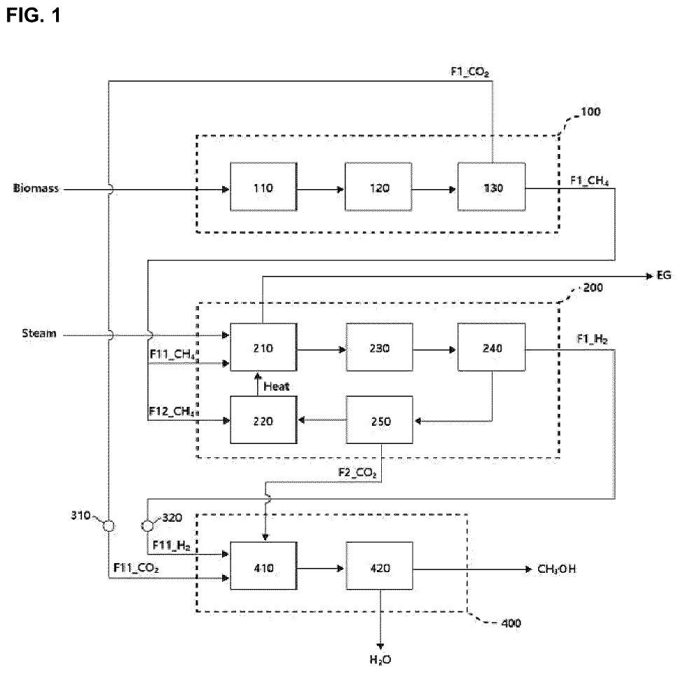

FIG. 1 is a view schematically illustrating the structure of a methanol synthesis system, according to an embodiment of the present disclosure. Referring to FIG. 1, according to an embodiment of the present disclosure, the methanol synthesis system may include a first producing device 100 to produce methane and carbon dioxide by introducing biomass, a second producing device 200 to produce hydrogen and carbon dioxide by allowing methane (that is, methane supplied through a first methane supply flow (F1_CH₄)), which is supplied from the first producing device 100, to react with steam, a first controller 310 to control a mole flow rate of carbon dioxide supplied from the first producing device 100, a second controller 320 to control a mole flow rate of hydrogen (that is, hydrogen supplied through a first hydrogen supply flow (F1_H₂)) supplied from the second producing device 200, and a methanol synthesizing device 400 to synthesize methanol by allowing carbon dioxide (that is, carbon dioxide supplied through a (1-1)-th carbon dioxide supply flow (F11_CO₂)) supplied from the first controller 310, hydrogen (that is, hydrogen supplied through a (1-1)-th hydrogen supply flow (F11_H₂)) supplied from the second controller 320, and carbon dioxide (that is, carbon dioxide (CO₂) supplied through a second carbon dioxide supply flow (F2_CO₂)) supplied from the second producing device 200 to react with each other.

Hereinafter, components of the methanol synthesis system according to an embodiment of the present disclosure will be described with reference to FIG. 1.

### 1. First Producing Device

According to an embodiment of the present disclosure, the first producing device 100 may produce methane and carbon dioxide by using biomass. The first producing device 100 may include an anaerobic digestion device 110 to produce biogas by performing an anaerobic digestion process for the biomass, a preprocessor 120 to remove foreign substances from the biogas, and a first separator 130 to separate methane and carbon dioxide from biogas having no foreign substances, as the foreign substances are removed from the biogas in the preprocessor 120.

According to an embodiment of the present disclosure, the biomass may refer to an unfertilized substance, such as an agricultural waste, manure, a household waste, a plant material, sewage, a green waste, or a food waste.

According to an embodiment of the present disclosure, the anaerobic digestion device 110 may produce the biogas by decomposing the biomass through the anaerobic digestion process. The anaerobic digestion process is to decompose the biomass by anaerobic microorganism.

According to an embodiment of the present disclosure, the anaerobic digestion device 110 may be maintained at the temperature ranging from 30° C. to 60° C. to activate the aerobic microorganism.

According to an embodiment of the present disclosure, the biogas produced from the anaerobic digestion device 110 may contain 60% to 70% of methane, and 30% to 40% of carbon dioxide.

According to an embodiment of the present disclosure, the preprocessor 120 may remove the foreign substance (e.g., non-biogas component), such as hydrogen sulfide (H₂S), siloxane, and ammonia from the biogas produced from the anaerobic digestion device 110. According to an embodiment of the present disclosure, the preprocessor 120 may include a filter to remove moisture, a sodium hydroxide (NaOH) absorber or an iron oxide absorber to remove a hydrogen sulfide, an activated carbon to remove siloxane, and/or a chelate resin to remove ammonia.

According to an embodiment of the present disclosure, the first separator 130 may separate methane and carbon dioxide from the biogas having no foreign substance, as the foreign substance is removed from the biogas in the preprocessor 120.

According to an embodiment of the present disclosure, the first separator 130 may separate methane and carbon dioxide from the biogas, which has foreign substance, as the foreign substance is removed from the biogas in the preprocessor 120, through a membrane separation manner using the difference in transmittance between methane gas and carbon dioxide gas.

According to an embodiment of the present disclosure, the methane separated in the first separator 130 may have the purity of at least 95%.

According to an embodiment of the present disclosure, methane separated in the first separator 130 may be transferred to the second producing device 200 through the first methane supply flow (F1_CH₄), and carbon dioxide separated in the first separator 130 may be transferred to the first controller 130 through the first carbon dioxide supply flow (F1_CO₂).

### 2. Second Producing Device

According to an embodiment of the present disclosure, the second producing device 200 may produce hydrogen by allowing methane, which is supplied through the first methane supply flow (FL_CH₄)), to react with steam. The second producing device 200 may include a reforming reactor 210 to allow steam and a portion (that is, methane supplied through the (1-1)-th methane supply flow (FL_CH₄)) of methane supplied through the first methane supply flow (FL_CH₄)) to react with each other, a heat supplier 220 to supply heat to the reforming reactor 210 by using, as fuel, a remaining portion (that is, methane supplied through the (1-2)-th methane supply flow (F12_CH₄)) of the methane supplied through the first methane supply flow (FL_CH₄)), a shift converter 230 to produce steam reforming gas by allowing the product from the reforming reactor 210 to react with steam, a second separator 240 to separate hydrogen from the steam reforming gas, and a third separator 250 to separate carbon dioxide from the steam reforming gas that is depleted in hydrogen after its separation from the steam reforming gas in the second separator 240.

According to an embodiment of the present disclosure, the first methane supply flow (F1_CH₄) for transferred methane, which is supplied from the first producing device 100, to the second producing device 200, may be divided into the (1-1)-th methane supply flow (F11_CH₄) introduced into the reforming reactor 210 and the (1-2)-th methane supply flow (F12_CH₄) introduced in to the heat supplier 220.

According to an embodiment of the present disclosure, the reforming reactor 210 may perform a steam methane reform (SMR) process, and the shift converter 230 may perform a water gas shift (WGS) process.

According to an embodiment of the present disclosure, the reforming reactor 210 may produce carbon monoxide (CO) and hydrogen by allowing methane, which is supplied through the (1-1)-th methane supply flow (F11_CH₄) to react with steam, through following Chemical Equation 1.

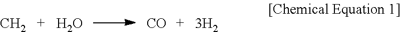

According to an embodiment of the present disclosure, the reaction through Chemical Equation 1 may be made inside the reforming reactor 210, in the presence of a catalyst such as a nickel catalyst.

According to an embodiment of the present disclosure, a strong endothermic reaction is made through Chemical Equation 1. To minimize the side reaction, the internal temperature of the reforming reactor 210 may be maintained to be 800° C. or more when making the reaction in Chemical Equation 1. To maintain the internal temperature of the reforming reactor 210 to be 800° C. or more, the heat supplier 220 may be used. The thermal energy derived from the heat supplier 220 may be discharged in the form of exhaust (EG) through the reforming reactor 210. The exhaust (EG) may include carbon dioxide.

According to an embodiment of the present disclosure, the remaining portion (that is, the (1-2)-th methane supply flow (F12_CH₄)) of methane supplied through the first methane supply flow (F1_CH₄) may be used as fuel to operate the heat supplier, thereby maximizing energy efficiency of the methanol synthesis system.

According to an embodiment of the present disclosure, the shift converter 230 may produce carbon dioxide and hydrogen (that is, steam reforming gas) by allowing carbon monoxide, which is produced from the reforming reactor 210, to react with steam.

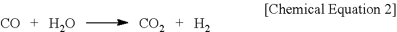

According to an embodiment of the present disclosure, the reaction in Chemical Equation 2 may be divided into a high temperature shift reaction made at the temperature ranging from 300° C. to 400° C. and a low temperature shift reaction made at the temperature ranging from 200° C. to 250° C.

According to an embodiment of the present disclosure, the whole chemical equation of the steam reforming reaction made in the reforming reactor 210 and the shift converter 230 may be expressed as in following Chemical Equation 3.

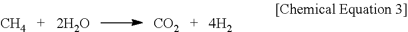

According to an embodiment of the present disclosure, the second separator 240 may separate hydrogen from the steam reforming gas produced from the reforming reactor 210 and the shift converter 230.

According to an embodiment of the present disclosure, the separation of hydrogen performed in the second separator 240 may be performed through a pressure swing adsorption (PSA) process, and the hydrogen separated in the second separator 240 may have the purity of at least 99%.

According to an embodiment of the present disclosure, hydrogen separated from the steam reforming gas may be transferred to the second controller 320 through a first hydrogen supply flow (F1_H₂), and steam reforming gas, which is depleted in hydrogen following the separation in the second separator 240 separated, may be transferred to the third separator 250.

According to an embodiment of the present disclosure, the third separator 250 may separate carbon dioxide from the hydrogen-depleted steam reforming gas supplied from the second separator 240, e.g., through a membrane separation process using the difference in gas permeability.

According to an embodiment of the present disclosure, the carbon dioxide separated in the third separator 250 may have the purity of at least 95%.

According to an embodiment of the present disclosure, the carbon dioxide separated in the third separator 250 may be transferred to the methanol synthesizing device 400 through a second carbon dioxide supply flow (F2_CO₂).

### 3. First Controller and Second Controller

According to an embodiment of the present disclosure, the first controller 310 and the second controller 320 may optimize a reaction by controlling the flow rates (specifically, mole flow rates) of carbon dioxide and hydrogen serving as source materials for synthesizing methanol in the methanol synthesizing device 400.

According to an embodiment of the present disclosure, the first controller 310 may control the flow rate (specifically, a mole flow rate) of carbon dioxide which is supplied from the first producing device 100 to the methanol synthesizing device 400. Accordingly, the carbon dioxide transferred through a first carbon dioxide supply flow (F1_CO₂) may be controlled in mole flow rate through the first controller 310, and then transferred to the methanol synthesizing device 400 through a (1-1)-th carbon dioxide supply flow (F11_CO₂), in which the mole flow rate of the carbon dioxide is controlled. In other words, the (1-1)-th carbon dioxide supply flow (F11_CO₂) may indicate that the mole flow rate in the first carbon dioxide supply flow (F1_CO₂) is controlled by the first controller 310.

According to an embodiment of the present disclosure, the second controller 320 may control the flow rate (specifically, a mole flow rate) of hydrogen supplied from the second producing device 200 to the methanol synthesizing device 400. Accordingly, the hydrogen transferred through a first hydrogen supply flow (F1_H₂) may be controlled in mole flow rate through the second controller 320, and then transferred to the methanol synthesizing device 400 through a (1-1)-th hydrogen supply flow (F11_H₂), in which the mole flow rate of the hydrogen is controlled. In other words, the (1-1)-th hydrogen supply flow (F11_H₂) may indicate that the mole flow rate in the first hydrogen supply flow (F1_H₂) is controlled by the second controller 320.

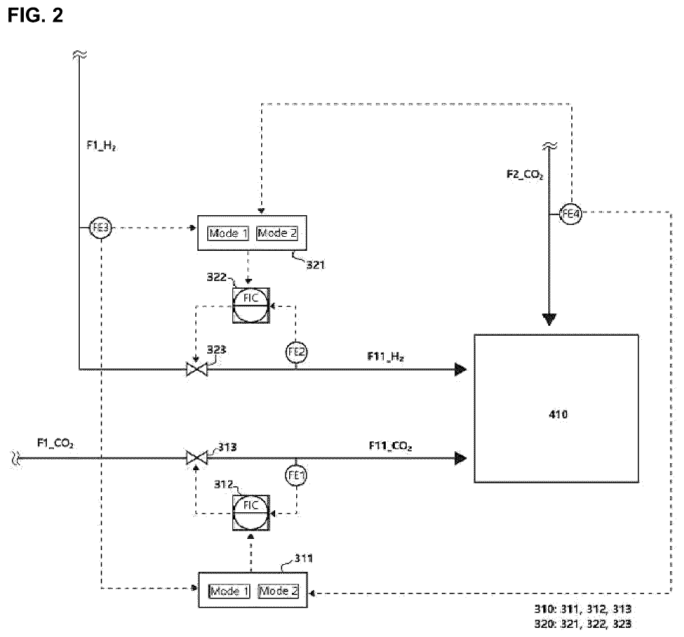

FIG. 2 is a view schematically illustrating the structure of a first controller and a second controller in a methanol synthesis system, according to an embodiment of the present disclosure. Hereinafter, the structure of each of the first controller 310 and the second controller 320 and a control pattern in a first mode and a second mode will be described with reference to FIG. 2.

Referring to FIG. 2, according to an embodiment of the present disclosure, the first controller 310 may include a first control mode selecting device 311, a first flow indictor controller (FIC) 312, and a first flow rate control valve 313.

According to an embodiment of the present disclosure, the first control mode selecting device 311 may determine a control mode of the first controller 310. Specifically, a user may select a first control mode (Mode 1) or a second control mode (Mode 2) as a control mode of the first controller 310, through the first control mode selecting device 311.

According to an embodiment of the present disclosure, when the first control mode 310 is controlled in the first mode (Mode 1), the mole flow rate of carbon dioxide, which is transferred through the first carbon dioxide supply flow (F1_CO₂), is controlled to the mole flow rate of carbon dioxide transferred through the (1-1)-th carbon dioxide supply flow (F11_CO₂) by the first controller 310. The mole flow rate of carbon dioxide transferred through the (1-1)-th carbon dioxide supply flow (F11_CO₂) may be determined through following Equation 1.

$$[ {Feed}_{{CO}_{2}} ] = \frac{[ {H}_{2} ]}{[ Eq ]} - [ C {O}_{2} ] \qquad Equation 1$$

In Equation 1 above, [Feed_{CO}_{₂}] may denote the mole flow rate of carbon dioxide supplied to the methanol synthesizing device 400, that is, the mole flow rate of carbon dioxide transferred through the (1-1)-th carbon dioxide supply flow (F11_CO₂), [H₂] may denote the mole flow rate of hydrogen supplied from the second producing device 200, that is, the mole flow rate of hydrogen transferred through the first hydrogen supply flow (F1_H₂), [CO₂] may denote the mole flow rate of carbon dioxide supplied from the second producing device 200, that is, the mole flow rate of carbon dioxide transferred through the second carbon dioxide supply flow (F2_CO₂), and [Eq] may denote an arbitrary value input by the user, and be the stoichiometric ratio between hydrogen and carbon dioxide necessary for optimally synthesizing methanol.

According to an embodiment of the present disclosure, the first control mode selecting device 311 may set the mol flow rate (that is, [Feed_{CO}_{₂}]) of carbon dioxide necessary for optimally synthesizing methanol, by substituting, into Equation 1, the mole flow rate in the first hydrogen supply flow (F1_H₂), which is measured through a third flow meter (FE3), the mole flow rate in the second carbon dioxide supply flow (F2_CO₂), which is measured through a fourth flow meter (FE4), and the arbitrary value (that is, [Eq]) input by the user

According to an embodiment of the present disclosure, the first FIC 312 may control the first flow rate control valve 313 by feed backing the mole flow rate in the (1-1)-th carbon dioxide supply flow (F11_CO₂), which is measured through the first flow meter FE1, based on a proper mole flow rate (that is, [Feed_{CO}_{₂}]) of carbon dioxide set by the first control mode selecting device 311, such that the mole flow rate in the (1-1)-th carbon dioxide supply flow (F11_CO₂) becomes the proper mole flow rate (that is, [Feed_{CO}_{₂}]) of carbon dioxide set by the first control mode selecting device 311. Accordingly, a side reaction may be prevented from being caused, as carbon dioxide is supplied in a smaller amount than that of carbon dioxide necessary for optimally synthesizing methanol in the methanol synthesizing device 400. The details thereof will be described below.

According to an embodiment of the present disclosure, when the first controller 310 is controlled in the second mode (Mode 2), the mole flow rate of carbon dioxide transferred through the (1-1)-th carbon dioxide supply flow (F11_CO₂) is prevented by the first controller 310, such that the mole flow rate of carbon dioxide transferred through the (1-1)-th carbon dioxide supply flow (F11_CO₂) may be measured as the value of ‘0’. In other words, when the first controller 310 is controlled in the second mode (Mode 2), carbon dioxide may not be supplied to the methanol synthesizing device 400 through the first carbon dioxide supply flow (F1_CO₂). Accordingly, the side reaction may be prevented from being caused, as carbon dioxide is supplied in a smaller amount than that of carbon dioxide necessary for optimally synthesizing methanol in the methanol synthesizing device 400. The details thereof will be described below.

Referring to FIG. 2, according to an embodiment of the present disclosure, the second controller 320 may include a second control mode selecting device 321, a second flow indictor controller (FIC) 322, and a second flow rate control valve 323.

According to an embodiment of the present disclosure, the second control mode selecting device 321 may determine a control mode of the second controller 320. Specifically, a user may select the first control mode (Mode 1) or the second control mode (Mode 2) as a control mode of the second controller 320, through the second control mode selecting device 321.

According to an embodiment of the present disclosure, when the second controller 320 is controlled in the first mode (Mode 1), the mole flow rate of hydrogen, which is transferred through the first hydrogen supply flow (F1_H₂), may be supplied to the methanol synthesizing device 400 without the control of the mole flow rate of hydrogen. Specifically, when the second controller 320 is controlled in the first mode (Mode 1), the flow rate of hydrogen supplied to the methanol synthesizing device 400 may be determined to the whole mole flow rate of hydrogen transferred through the first hydrogen supply flow (F1_H₂). Accordingly, the mole flow rate of hydrogen through the first hydrogen supply flow (F1_H₂), which is measured through the third flow meter (FE3), may be equal to the mole flow rate of hydrogen transferred through the (1-1)-th hydrogen supply flow (F11_H₂). Accordingly, a side reaction may be prevented from being caused, as carbon dioxide is supplied in a smaller amount than that of carbon dioxide necessary for optimally synthesizing methanol in the methanol synthesizing device 400. The details thereof will be described below.

According to an embodiment of the present disclosure, when the second controller 320 is controlled in the second mode (Mode 2), the mole flow rate of hydrogen transferred through the first hydrogen supply flow (F1_H₂) may be controlled to the mole flow rate of hydrogen transferred through the (1-1)-th hydrogen supply flow (F11_H₂) by the second controller 320. The mole flow rate of hydrogen transferred through the (1-1)-th hydrogen supply flow (F11_H₂) may be determined through Equation 2.

$$[ {Feed}_{{H}_{2}} ] = [ C {O}_{2} ] \times [ Eq ] \qquad Equation 2$$

In Equation 2 above, [Feed_{H}_{₂}] may denote the mole flow rate of hydrogen supplied to the methanol synthesizing device 400, that is, the mole flow rate of hydrogen (H₂) transferred through the (1-1)-th hydrogen supply flow (F11_H₂), 662 may denote the mole flow rate of carbon dioxide supplied from the second producing device 200, and [Eq] may denote an arbitrary value input by the user, and the stoichiometric ratio between hydrogen and carbon dioxide necessary for optimally synthesizing methanol.

According to an embodiment of the present disclosure, the second control mode selecting device 321 may set the mol flow rate (that is, Feed_{H}_{₂}) of hydrogen necessary for optimally synthesizing methanol, by substituting, into Equation 2, the mole flow rate of hydrogen transferred through the first hydrogen supply flow (F1_H₂), which is measured through the third flow meter (FE3), the mole flow rate of carbon dioxide transferred through the second carbon dioxide supply flow (F2_CO₂), which is measured through the fourth flow meter (FE4), and the arbitrary value (that is, [Eq]) input by the user

According to an embodiment of the present disclosure, the second FIC 322 may control the second flow rate control valve 323 by feed backing the mole flow rate of hydrogen transferred through the (1-1)-th hydrogen supply flow (F11_H₂), which is measured through the second flow meter FE2, based on a proper mole flow rate (that is, [Feed_{H}_{₂}]) of hydrogen set by the second control mode selecting device 321, such that the mole flow rate of hydrogen transferred through the (1-1)-th hydrogen supply flow (F11_H₂) becomes the proper mole flow rate (that is, [Feed_{H}_{₂}]) of hydrogen set by the second control mode selecting device 321. Accordingly, the side reaction may be prevented from being caused, as carbon dioxide is supplied in a smaller amount than that of carbon dioxide necessary for optimally synthesizing methanol in the methanol synthesizing device 400. The details thereof will be described below.

According to an embodiment of the present disclosure, the same control mode may be identically applied to the first controller 310 and the second controller 320. Specifically, when the first controller 310 is controlled in the first mode (Mode 1), even the second controller 320 may be controlled in the first mode (Mode 1). When the first controller 310 is controlled in the second mode (Mode 2), even the second controller 320 may be controlled in the second mode (Mode 2).

Accordingly, when the first controller 310 and the second controller 320 are controlled in the first mode (Mode 1), the mole flow rate of carbon dioxide, which is controlled by the first controller 310, may be determined through Equation 1, and the flow rate of hydrogen controlled by the second controller 320 may be determined to the whole mole flow rate of hydrogen supplied from the second producing device 200. In addition, when the first controller 310 and the second controller 320 are controlled in the second mode (Mode 2), the mole flow rate of carbon dioxide controlled by the first controller 310 may be determined to the value of ‘0’, and the mole flow rate of hydrogen controlled by the second controller 320 may be determined through Equation 2.

### 4. Methanol Synthesizing Device

According to an embodiment of the present disclosure, the methanol synthesizing device 400 may synthesize methanol by allowing carbon dioxide supplied from the first controller 310, hydrogen supplied from the second controller 320, and carbon dioxide supplied from the second producing device 200 to react with each other.

According to an embodiment of the present disclosure, the methanol synthesizing device 400 may include a synthesis reactor 410 to make synthesis-reaction of methanol and a fourth separator 420 to separate methanol, which is produced from the synthesis reactor 410, and water.

According to an embodiment of the present disclosure, the synthesis-reaction of methanol through following Chemical Equation 4 may be made in the synthesis reactor 410.

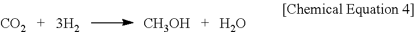

According to an embodiment of the present disclosure, when the synthesis-reaction of methanol is made according to Chemical Equation 4, the stoichiometric ratio between hydrogen and carbon dioxide necessary for optimally synthesizing methanol (or the stoichiometric ratio between hydrogen to carbon dioxide necessary for optimally synthesizing methanol) may be ‘3’. In other words, the mole flow rate of hydrogen necessary for optimally synthesizing methanol should be three times the mole flow rate of carbon dioxide. When the supply of hydrogen and carbon dioxide fails to satisfy the stoichiometric ratio between hydrogen and carbon dioxide necessary for optimally synthesizing methanol, the side reaction may be caused such that methanol is not smoothly supplied. For example, the side reaction is to produce methane, as carbon dioxide is supplied in a smaller amount.

Meanwhile, the whole Chemical Equation for a steam reforming reaction made in the reforming reactor 210 may be expressed as in Chemical Equation 3 as described above. Theoretically, four mole of hydrogen may be produced for one mole of carbon dioxide. Accordingly, hydrogen supplied from the second producing device 200 is supplied above the range necessary for optimizing a methanol synthesis reaction through Chemical Equation 4, while carbon dioxide supplied from the second producing device 200 is supplied below the range necessary for optimizing a methanol synthesis reaction through Chemical Equation 4. In this case, the stoichiometric ratio between hydrogen and carbon dioxide supplied from the second producing device 200 fails to satisfy the stoichiometric ratio (or the stoichiometric ratio of hydrogen to carbon dioxide) between hydrogen and carbon dioxide necessary for optimize the methanol synthesis reaction through Chemical Equation 4. This may be recognized through mole flow rate in the (1-1)-th methane supply flow (F11_CH₄), the mole flow rate of steam supplied through the second producing device 200, and mole flow rates in the first hydrogen supply flow (F1_H₂), and the second carbon dioxide supply flow (F2_CO₂), which are measured when actually performing processes, as shown in Table 1.

TABLE 1

| | F11_CH4 | Steam | F1_H2 | F2_OO2 |
|---|---|---|---|---|
| Mole flow | 0.62 | 187 | 1.72 | 0.51 |
| rate (kmol/h) | | | | |

Referring to Table 1, it may be recognized that the flow rate in the first hydrogen supply flow (F1_H₂) which is a supply flow of hydrogen produced from the second producing device 200 exceeds three times of the flow rate in the second carbon dioxide supply flow F2_CO₂ which is a supply flow of carbon dioxide produced from the second producing device 200. Accordingly, when hydrogen and carbon dioxide produced from the second producing device 200 are supplied to the methanol synthesizing device 400 without limitation of flow rates of hydrogen and carbon dioxide, the side reaction may be caused, which is similarly to when carbon dioxide supplied from the second producing device 200 is supplied below the range necessary for optimizing a methanol synthesis reaction through Chemical Equation 4.

According to an embodiment of the present disclosure, the methanol synthesis system may optimize a reaction, as the first controller 310 and the second controller 320 control the flow rates (specifically, mole flow rates) of carbon dioxide and hydrogen serving as source materials for synthesizing methanol in the methanol synthesizing device 400, thereby preventing the above-described side reaction from being caused. For example, when the methanol synthesis reaction made in the synthesis reactor 410 follows Chemical Equation 4, the first controller 310 and the second controller 320 may be controlled in the first mode (Mode 1) or the second mode (Mode 2). In this case, the value of [Eq] may be selected in the range from 3 to 6.

According to an embodiment of the present disclosure, the reaction in Chemical Equation 4 may be performed at the temperature of 250° C. under at least 50 atmospheric pressure in presence of copper catalyst, or be performed at the temperature of 150° C. under at least 30 atmospheric pressure in presence of platinum catalyst.

### <Method for Controlling Methanol Synthesis System>

The present disclosure provides a method for controlling a methanol synthesis system.

According to an embodiment of the present disclosure, the method for controlling the methanol synthesis system is to control the methanol synthesis system in the first mode, in which the mole flow rate of carbon dioxide, which is controlled by the first controller, through Equation 1, is determined, and the mole flow rate of hydrogen controlled by the second controller is determined to the whole mole flow rate of hydrogen supplied from the second producing device, or in the second mode, in which the mole flow rate of carbon dioxide controlled by the first controller is determined to the value of ‘0’, and the mole flow rate of hydrogen controlled by the second controller is determined through Equation 2.

$$[ {Feed}_{{CO}_{2}} ] = \frac{[ {H}_{2} ]}{[ Eq ]} - [ C {O}_{2} ] \qquad Equation 1$$

In Equation 1 above, [Feed_{CO}_{₂}] may denote the mole flow rate of carbon dioxide supplied to the methanol synthesizing device 400, that is, the mole flow rate of carbon dioxide transferred through the (1-1)-th carbon dioxide supply flow (F11_CO₂), [H₂] may denote the mole flow rate of hydrogen supplied from the second producing device 200, that is, the mole flow rate of hydrogen transferred through the first hydrogen supply flow (F1_H₂), [CO₂] may denote the mole flow rate of carbon dioxide supplied from the second producing device 200, that is, the mole flow rate of carbon dioxide transferred through the second carbon dioxide supply flow (F2_CO₂), and [Eq] may denote an arbitrary value input by the user, and be the stoichiometric ratio between hydrogen and carbon dioxide necessary for optimally synthesizing methanol.

$$[ {Feed}_{{H}_{2}} ] = [ C {O}_{2} ] \times [ Eq ] \qquad Equation 2$$

In Equation 2 above, [Feed_{H}_{₂}] may denote the mole flow rate of hydrogen supplied to the methanol synthesizing device 400, that is, the mole flow rate of hydrogen (H₂) transferred through the (1-1)-th hydrogen supply flow (F11_H₂), [CO₂] may denote the mole flow rate of carbon dioxide supplied from the second producing device 200, and [Eq] may denote an arbitrary value input by the user, and be the stoichiometric ratio between hydrogen and carbon dioxide necessary for optimally synthesizing methanol.

According to an embodiment of the present disclosure, when the methanol synthesizing device 400 synthesizes methanol by allowing carbon dioxide supplied from the first controller, hydrogen supplied from the second controller, and carbon dioxide supplied from the second producing device to react with each other through following Chemical Equation 4, [Eq] may be in the range 3 to 6 in Equations 1 and 2.

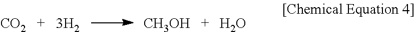

According to an embodiment of the present disclosure, the same control mode may be identically applied to the first controller 310 and the second controller 320. Specifically, when the first controller 310 is controlled in the first mode (Mode 1), even the second controller 320 may be controlled in the first mode (Mode 1). When the first controller 310 is controlled in the second mode (Mode 2), even the second controller 320 may be controlled in the second mode (Mode 2).

Hereinafter, the details of the first mode (Mode 1) and the second mode (Mode 2) will be described, in the method for controlling the methanol synthesis system according to an embodiment of the present disclosure, on the condition that the methanol synthesizing device 400 synthesizes methanol by allowing carbon dioxide supplied from the first controller 310, hydrogen supplied from the second controller 320, and carbon dioxide supplied from the second producing device 200 to react with each other through following Chemical Equation 4

### 1. First Mode

According to an embodiment of the present disclosure, when hydrogen and carbon dioxide produced from the second producing device 200 are transferred to the methanol synthesizing device 400 to perform the methanol synthesis reaction without the additional control over the flow rates of hydrogen and carbon dioxide, the side reaction may be caused, which is similarly to when carbon dioxide supplied from the second producing device 200 is supplied below the range necessary for optimizing the methanol synthesis reaction through Chemical Equation 4, as described above. Accordingly, the methanol synthesis system according to an embodiment of the present disclosure is controlled in the first mode (Mode 1) to additionally supply carbon dioxide, which is produced from the first producing device 100, to the methanol synthesizing device 400, thereby satisfying the stoichiometric ratio of hydrogen to carbon dioxide necessary for optimizing the methanol synthesis reaction through Chemical Equation 4, such that the side reaction is prevented from being caused.

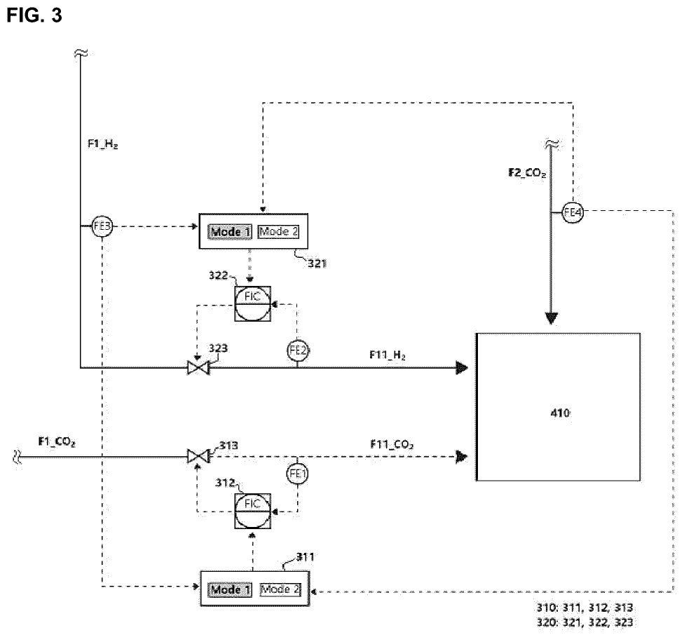

FIG. 3 is a view illustrating a methanol synthesis system controlled in a first mode, according to an embodiment of the present disclosure. Referring to FIG. 3, when the methanol synthesis system according to an embodiment of the present disclosure is controlled in the first mode (Mode 1), the mole flow rate of carbon dioxide controlled by the first controller 310 may be determined through Equation 1, and the flow rate of hydrogen controlled by the second controller 320 may be determined to the whole mole flow rate of hydrogen supplied from the second producing device 200.

Accordingly, the mole flow rate of carbon dioxide necessary for optimizing the methanol synthesis reaction may be complemented, thereby preventing the side reaction from being caused, and carbon dioxide produced from the first producing device 100 may be recycled, thereby reducing a green-house effect resulting from emission of carbon dioxide.

### 2. Second Mode

According to an embodiment of the present disclosure, when hydrogen and carbon dioxide produced from the second producing device 200 are transferred to the methanol synthesizing device 400 without the additional control over the flow rates of hydrogen and carbon dioxide to perform the methanol synthesis reaction, the side reaction may be caused, which is similarly to when hydrogen supplied from the second producing device 200 is supplied above the range necessary for optimizing the methanol synthesis reaction through Chemical Equation 4, as described above. Accordingly, the methanol synthesis system according to an embodiment of the present disclosure is controlled in the second mode (Mode 2) to reduce the flow rate of hydrogen produced from the second producing device 200, thereby satisfying the stoichiometric ratio of hydrogen to carbon dioxide necessary for optimizing the methanol synthesis reaction through Chemical Equation 4, such that the side reaction is prevented from being caused.

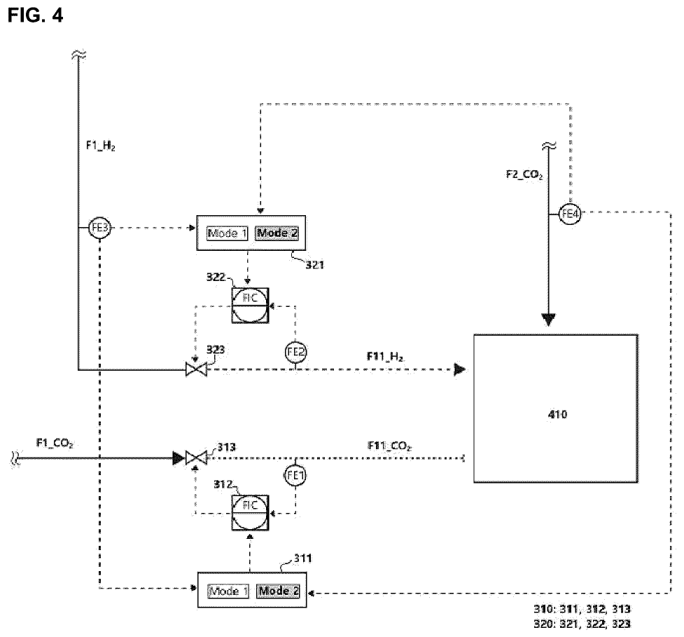

FIG. 4 is a view illustrating a methanol synthesis system controlled in a second mode, according to an embodiment of the present disclosure. Referring to FIG. 4, when the methanol synthesis system according to an embodiment of the present disclosure is controlled in the second mode (Mode 2), the mole flow rate of carbon dioxide controlled by the first controller 310 may be determined to the value of ‘O’, and the flow rate of hydrogen controlled by the second controller 320 may be determined through Equation 2.

Accordingly, the mole flow rate of hydrogen necessary for optimizing the methanol synthesis reaction may be reduced, thereby preventing the side reaction from being caused.

According to an embodiment of the present disclosure, the methanol synthesis system may control the optimal condition for synthesizing methanol (CH₃OH) while using biomass.

According to an embodiment of the present disclosure, in the method for controlling the methanol synthesis system, the optimal condition for synthesizing methanol may be controlled while using biomass.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims.

What is claimed is:

1. A methanol synthesis system comprising:
a first producing device configured to produce methane and carbon dioxide by introducing biomass;
a second producing device configured to produce hydrogen and carbon dioxide by allowing the methane, which is supplied from the first producing device, to react with steam;
a first controller configured to control a mole flow rate of the carbon dioxide supplied from the first producing device;
a second controller configured to control a mole flow rate of the hydrogen supplied from the second producing device; and
a methanol synthesizing device configured to synthesize methanol by reacting the carbon dioxide supplied from the first controller, the hydrogen supplied from the second controller, and the carbon dioxide supplied from the second producing device.

2. The methanol synthesis system of claim 1, wherein the first producing device includes:
an anaerobic digestion device configured to produce biogas, comprising methane and carbon dioxide, by performing an anaerobic digestion process on the biomass;
a preprocessor configured to remove a foreign substance from the biogas to produce a processed biogas; and
a first separator configured to separate the methane and the carbon dioxide from the processed biogas.

3. The methanol synthesis system of claim 2, wherein the methane separated in the first separator has purity of at least 95%.

4. The methanol synthesis system of claim 1, wherein the second producing device includes:
a reforming reactor configured to allow the steam and a first portion of the methane supplied from the first producing device to react with each other to form a product gas;
a heat supplier configured to supply heat to the reforming reactor by using, as fuel, a second portion of the methane supplied from the first producing device;
a shift converter configured to produce steam reforming gas, comprising hydrogen and carbon dioxide, by reacting the product gas from the reforming reactor with the steam;
a second separator configured to separate hydrogen from the steam reforming gas to provide hydrogen and a hydrogen depleted steam reforming gas; and
a third separator configured to separate carbon dioxide from the hydrogen depleted steam reforming gas.

5. The methanol synthesis system of claim 4, wherein the product gas produced in the reforming reactor comprises carbon monoxide and hydrogen and is generated under reaction conditions that allow the portion of the methane supplied from the first producing device and the steam to react according to Chemical Equation 1, 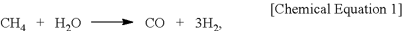
and
wherein the steam reforming gas produced in the shift converter comprises carbon dioxide and hydrogen and is generated under reaction conditions that allow the carbon monoxide and the hydrogen produced from the reforming reactor to react according to Chemical Equation 2,

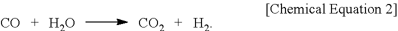

6. The methanol synthesis system of claim 4, wherein the hydrogen separated from the steam reforming gas in the second separator has purity of at least 99%.

7. The methanol synthesis system of claim 1, wherein a mole flow rate of the carbon dioxide supplied from the first producing device to the methanol synthesizing device and a mole flow rate of the hydrogen supplied from the second producing device to the methanol synthesizing device are controlled in a first mode, and
wherein the first mode operates to determine the mole flow rate of the carbon dioxide controlled by the first controller through following Equation 1, and to determine a mole flow rate of the hydrogen controlled by the second controller to a whole mole flow rate of the hydrogen supplied from the second producing device,
$$[ {Feed}_{{CO}_{2}} ] = \frac{[ {H}_{2} ]}{[ Eq ]} - [ C {O}_{2} ] , \qquad [ Equation 1 ]$$
wherein,
[Feed_{CO}_{₂}] denotes the mole flow rate of the carbon dioxide supplied to the methanol synthesizing device,
[H₂] denotes the mole flow rate of the hydrogen supplied from the second producing device,
[CO₂] denotes the mole flow rate of the carbon dioxide supplied from the second producing device, and
[Eq] denotes an arbitrary value input by a user.

8. The methanol synthesis system of claim 7, wherein the methanol synthesizing device synthesizes the methanol by allowing the carbon dioxide supplied from the first controller, the hydrogen supplied from the second controller, and the carbon dioxide supplied from the second producing device to react with each other through Chemical Equation 4, 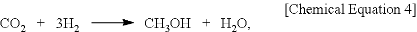
and
wherein [Eq] in Equation 1 is in a range 3 to 6.

9. The methanol synthesis system of claim 1, wherein the mole flow rate of the carbon dioxide supplied from the first producing device to the methanol synthesizing device and the mole flow rate of the hydrogen supplied from the second producing device to the methanol synthesizing device are controlled in a second mode, and
wherein the second mode operates to determine the mole flow rate of the carbon dioxide controlled by the first controller to a value of zero (‘0’), and to determine the mole flow rate of the hydrogen controlled by the second controller through following Equation 2,
$$[ {Feed}_{{H}_{2}} ] = [ C {O}_{2} ] \times [ Eq ] , \qquad [ Equation 2 ]$$
wherein,
[Feed_{CO}_{₂}] denotes the mole flow rate of the hydrogen supplied to the methanol synthesizing device,
[CO₂] denotes the mole flow rate of the carbon dioxide supplied from the second producing device, and
[Eq] denotes an arbitrary value input by a user.

10. The methanol synthesis system of claim 9, wherein the methanol synthesizing device synthesizes the methanol by allowing the carbon dioxide supplied from the first controller, the hydrogen supplied from the second controller, and the carbon dioxide supplied from the second producing device to react with each other through following Chemical Equation 4, 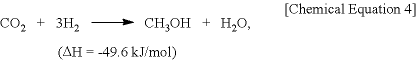
and
wherein [Eq] in Equation 1 is in a range 3 to 6.

11. The methanol synthesis system of claim 1, wherein the methanol synthesizing device synthesizes the methanol by allowing the carbon dioxide supplied from the first controller, the hydrogen supplied from the second controller, and the carbon dioxide supplied from the second producing device to react with each other through following Chemical Equation 4,

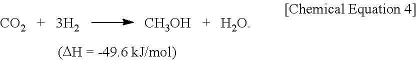

12. The methanol synthesis system of claim 11, wherein reaction in Chemical Equation 4 is performed at a temperature of 250° C. under at least 50 atmospheric pressure in presence of copper catalyst.

13. The methanol synthesis system of claim 11, wherein reaction in Chemical Equation 4 is performed at a temperature of 150° C. under at least 30 atmospheric pressure in presence of platinum catalyst.

14. A method for controlling the methanol synthesizing device according to claim 1, comprising controlling the methanol synthesizing device in either
in a first mode in which a mole flow rate of carbon dioxide, which is controlled by the first controller, is determined through following Equation 1, and a mole flow rate of hydrogen controlled by the second controller is determined to a whole mole flow rate of hydrogen supplied from the second producing device,
$$[ {Feed}_{{CO}_{2}} ] = \frac{[ {H}_{2} ]}{[ Eq ]} - [ C {O}_{2} ] , \qquad [ Equation 1 ]$$
wherein in Equation 1,
[Feed_{CO}_{₂}] denotes the mole flow rate of the carbon dioxide supplied to the methanol synthesizing device,
[H₂] denotes the mole flow rate of the hydrogen supplied from the second producing device,
[CO₂] denotes the mole flow rate of the carbon dioxide supplied from the second producing device, and
[Eq] denotes an arbitrary value input by a user; or
in a second mode, in which the mole flow rate of the carbon dioxide controlled by the first controller is determined to a value of zero (‘0’), and the mole flow rate of the hydrogen controlled by the second controller is determined through following Equation 2,
[Feed_{H}_{₂}]=[CO₂]×[Eq], [Equation 2]
wherein in Equation 2,
[Feed_{H}_{₂}] denotes the mole flow rate of the hydrogen supplied to the methanol synthesizing device,
[CO₂] denotes the mole flow rate of the carbon dioxide supplied from the second producing device, and
[Eq] denotes an arbitrary value input by a user.

15. The method of claim 14, wherein the methanol synthesizing device synthesizes the methanol by allowing the carbon dioxide supplied from the first controller, the hydrogen supplied from the second controller, and the carbon dioxide supplied from the second producing device to react with each other through following Chemical Equation 4, 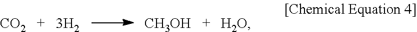
and
wherein [Eq] in Equations 1 and 2 is in a range 3 to 6.
